(19) United States
(12) Patent Application Publication
(10) Pub. No.: US 2025/0017562 A1
(43) Pub. Date: Jan. 16, 2025

(54) SYSTEM OF DETERMINING THE CORRECT IMPLANTABLE COLLAMER LENS SIZE FROM ULTRASOUND IMAGES OF THE EYE

(71) Applicant: Dan Z. Reinstein, London (GB)

(72) Inventors: Dan Z. Reinstein, London (GB); Ryan S. Vida, London (GB); Timothy James Archer, London (GB)

(21) Appl. No.: 18/895,284

(22) Filed: Sep. 24, 2024

Related U.S. Application Data
(63) Continuation of application No. 17/066,473, filed on Oct. 8, 2020, now Pat. No. 12,138,115.
(60) Provisional application No. 62/913,740, filed on Oct. 11, 2019.

(51) Int. Cl. A61B 8/10; A61B 8/08; A61F 2/16

(52) U.S. Cl. CPC A61B 8/10; A61B 8/5215; A61F 2/16

(57) ABSTRACT

A method for operating a data processing system to provide an estimate of a vault value for a PIOL to be inserted in a patient, a system for predicting the vault that will be realized for an implant, and computer readable medium that includes instructions that cause a data processing system to implement the method when the instructions are executed by a data processing system are disclosed. The method includes causing the data processing system to receive ultrasound-based anatomical measurements of the patient's eye, causing the data processing system to receive parameters specifying a power and size for the PIOL; and utilizing a calibrated model of a human eye to predict the vault based on the anatomical measurements and the parameters specifying the power and the size for the PIOL.

### RELATED APPLICATIONS

This application is a continuation of U.S. patent application Ser. No. 17/066,473, filed Oct. 8, 2020, for System of Determining the Correct Implantable Collamer Lens Size from Ultrasound Images of the Eye, which claims the benefit of U.S. Provisional Application 62/913,740 filed Oct. 11, 2019, both of which are incorporated herein by reference.

### BACKGROUND

A phakic intraocular lens (PIOL), such as the implantable collamer lens, is an attractive alternative for some patients seeking decreased dependence on glasses and/or contact lenses. The PIOL is inserted surgically between the patient's crystalline lens and the iris. The separation between the anterior surface of the patient's crystalline lens and the back of the PIOL is known as lens separation or “vault”. Selecting the correct size for the PIOL presents significant challenges. Problems may arise if the size of the PIOL implanted is too large (resulting in very high vault) or too small (resulting in vely low or no vault).

If the size of the implanted PIOL is too large and the resulting vault is too high then the PIOL may push the iris forward. This can lead to narrowing of the anterior chamber angle which may increase the risk for intraocular pressure changes with subsequent irreversible glaucomatous nerve damage. In addition, the back surface of the iris, the pigment epithelium of the iris, can be excessively rubbed/chaffed against by the oversized PIOL causing the pigment of the iris to rub off and disperse into the anterior chamber. Unwanted pigment cells in the anterior chamber may affect the trabecular meshwork causing increased fluid outflow resistance, an increase in intraocular pressure and glaucomatous nerve damage.

If the size of the implanted PIOL is too small and the resulting vault is too low; then the PIOL may come into contact with the anterior surface of the crystalline lens. This may result in early cataract formation. In addition, if the vault of the PIOL is too low (even if not touching the anterior surface of the crystalline lens), then it may interfere with the flow of nutrients to the crystalline lens which also may contribute to early cataract formation.

Selecting the correct PIOL lens size is complicated by two factors. First, these lenses are only available in discrete size increments. Second, the anatomical structures on which the PIOL haptics and footplates (“feet”) rest are not visible using optical methods (e.g. optical coherence tomography) because the iris is not transparent to light. Accordingly, the basic standard method for selecting the lens size is to use the horizontal white-to-white diameter, under the assumption that this is sufficiently correlated to the dimensions of the posterior chamber, the space behind the iris, to yield a reasonable and safe vault between the PIOL from the natural crystalline lens. However, multiple studies have shown that there is only a very weak correlation between white-to-white and the internal posterior chamber sulcus-to-sulcus distance. Therefore, various investigators have attempted to find optically visible measurements, such as the anterior chamber angle diameter, that can be used to improve the prediction to posterior chamber dimensions and improve upon lens size recommendations. These attempts have only been partially successful in predicting the vault values.

### BRIEF DESCRIPTION OF THE DRAWINGS

FIG. 1 is a cross-sectional view of a portion of an eye illustrating the structures that are relevant to a PIOL implant.

FIG. 2 illustrates the region of the eye shown in FIG. 1 after the insertion of a PIOL.

FIG. 3 is cross-sectional view of the anterior portion of a human eye.

FIG. 4 illustrates one embodiment of an imaging system according to the present disclosure.

### SUMMARY

The present invention includes a method for operating a data processing system to provide an estimate of a vault value for a PIOL to be inserted in a patient, a system for predicting the vault that will be realized for an implant, and computer readable medium that includes instructions that cause a data processing system to implement the method when the instructions are executed by a data processing system. The method includes:
causing the data processing system to receive ultrasound-based anatomical measurements of the patient's eye, causing the data processing system to receive parameters specifying a power and size for the PIOL; and utilizing a calibrated model of a human eye to predict the vault based on the anatomical measurements and the parameters specifying the power and the size for the PIOL and displaying said vault to a user of said data processing system to determine if said vault is satisfactory for said patient.

In one aspect, the data processing system receives the ultrasound-based anatomical measurements which include causing the data processing system to receive an ultrasound image of the patient's eye and determining the ultrasound-based anatomical measurements from the ultrasound image.

In one aspect, the anatomical measurements comprise measurements of the diameter of the plane passing through the ciliary body (CBID).

In one aspect, the anatomical measurements comprise measurements of the sulcus-to-sulcus-lens-rise (STSL).

In one aspect, the anatomical measurements comprise measurements of the scotopic pupil diameter (SPD).

In one aspect, the calibrated model is a linear model.

A system according to the present invention includes a data processing system and an ultrasound imaging device. The ultrasound imager is adapted for providing an image of a patient's eye, and the data processing system receiving a plurality of anatomical measurements of a patient's eye from the image, receiving parameters specifying a power and size for a PIOL; and providing a measurement of a vault for the patient's eye utilizing a calibrated model of a human eye to predict the vault based on the anatomical measurements and the parameters specifying a power and the a size for the PIOL.

In one aspect, the anatomical measurements comprise measurements of CBID.

In one aspect, the anatomical measurements comprise measurements of STSL.

In one aspect, the anatomical measurements comprise measurements of SPD.

The computer readable medium stores instructions that cause a data processing

system to execute a method for operating a data processing system to provide an estimate of a vault value for an ICL to be inserted in a patient. The method includes causing the data processing system to receive ultrasound-based anatomical measurements of the patient's eye; causing the data processing system to receive parameters specifying a power and size for the ICL; and utilizing a calibrated model of a human eye to predict the vault based on the anatomical measurements and the parameters specifying the power and the size for the ICL.

In one aspect, causing the data processing system to receive the ultrasound-based anatomical measurements includes causing the data processing system to receive an ultrasound image of the patient's eye and determining the ultrasound-based anatomical measurements from the ultrasound image.

In one aspect, the anatomical measurements comprise measurements of CBID.

In one aspect, the anatomical measurements comprise measurements of STSL.

In one aspect, the anatomical measurements comprise measurements of SPD.

In one aspect, the calibrated model is a linear model.

### DETAILED DESCRIPTION

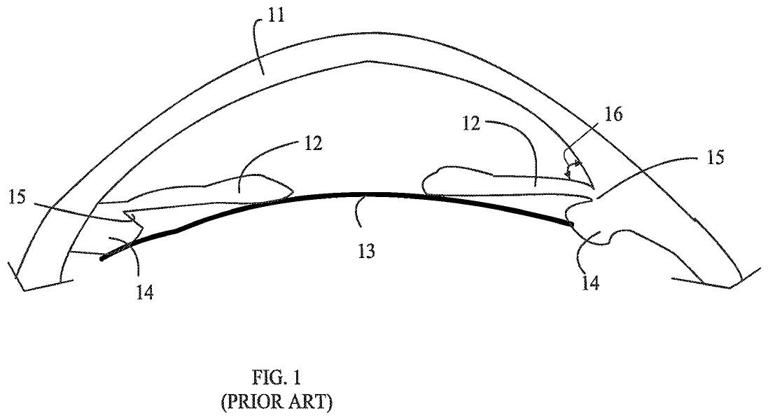

The manner in which the present invention provides its advantages can be more easily understood with reference to FIG. 1 which is a cross-sectional view of a portion of an eye illustrating the structures that are relevant to a PIOL implant. The cornea is shown at 11. The upper surface of the capsule containing the crystalline lens is shown at 13. The iris 12 rests on the upper surface of the capsule. The angle between the iris and the inner surface of the cornea is shown at 16. The ciliary body is shown at 14. The sulcus is shown at 15.

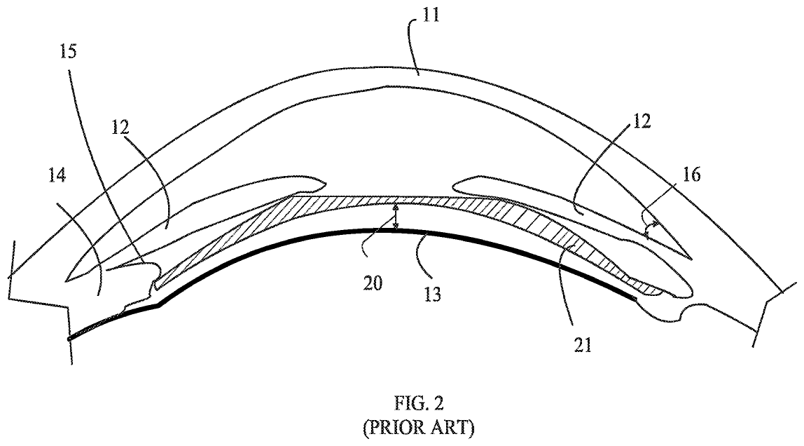

Refer now to FIG. 2, which illustrates the region of the eye shown in FIG. 1 after the insertion of a PIOL 21. The PIOL is inserted between the crystalline lens and the iris 12. The feet of the PIOL rest on the ciliary body. PIOL 21 vaults over crystalline lens capsule 13 as shown at 20. As a result, the portion of the iris nearest the pupil is moved towards the cornea. This results in the angle 16 decreasing. The greater the vault 20, the smaller the angle 16 becomes. In addition, the posterior surface of the iris 12 in contact with the PIOL 21 can rub in a manner which results in pigment being released from the iris. The higher the vault, the greater the abrasive forces that can lead to such a release.

If, on the other hand, the vault is too low, the PIOL can rub on the anterior surface of the crystalline lens capsule 13, which can lead to early cataract formation. In addition, if the vault is too low, the PIOL 21 can interfere with the nutrient flow across the surface of the capsule which may also contribute to early cataract formation.

The feet of the PIOL rest on the ciliary body between the capsule and the sulcus. Increasing the size of the PIOL increases the vault, since the PIOL must bend upward to accommodate the greater length.

The problem of predicting the vault for a given PIOL size is complicated by the fact that the eye parameters that can be directly measured by optical methods do not include direct measurements of structures behind the iris, since the iris blocks visible light. While there are correlations between the visibly measurable parameters and the structures behind the iris, the correlations are not sufficiently strong to provide the desired accuracy in some eyes.

One method for improving the prediction of the vault for a given PIOL size utilizes high-frequency ultrasound to image the eye. Since sound waves are not blocked by the optically opaque portions of the iris, direct measurements of the structures in the posterior chamber of the eye can be made.

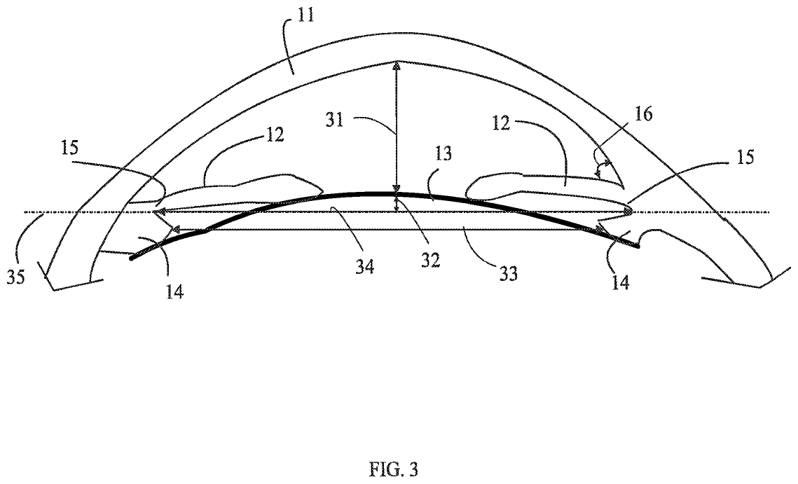

While methods for predicting the vault from measurements made with ultrasound imaging have been suggested, the accuracy with which the vault height is predicted is still insufficient for some purposes. The manner in which the methods that use ultrasound images for part of the parameters used to compute the vault for various lens sizes can be more easily understood with reference to FIG. 3, which is cross-sectional view of the anterior portion of a human eye. It should be noted that the eye is spherical, and hence, many of the structures are circular in conformation.

One easily identifiable “landmark” is the sulcus. The plane 35 passing through the sulcus is used to define three measurements of interest. The sulcus-to-sulcus distance (STS) is shown at 34. This is essentially the diameter of the ring of the sulcus. The maximum distance 32 from the plane defined by the sulcus to the anterior edge of the capsule will be referred to as the STSL. The distance 31 from the capsule to the anterior portion of the cornea will be referred to as the anterior chamber depth (ACD). The diameter 33 of the plane passing through the CBID is also predictive.

In addition to the parameters that depend on behind the iris imaging, it has been found that the SPD has predicative value in determining the vault or finding a lens size that provides the desired vault. The SPD is the diameter of the pupil under scotopic light conditions, and is related to PIOL vault due to the difference in force exerted by the iris onto the PIOL for small and large pupils. The pupil size varies according to lighting conditions such that in bright light the pupil constricts and in dim light it dilates. Therefore, depending on lighting conditions and according to how lighting changes during the day the pupil size will be different and produce a specific change in the separation between the PIOL and the crystalline lens. Scotopic pupil size is defined as the pupil diameter under scotopic lighting conditions, defined as ambient 0.04 lux. In addition, the lens size and PIOL power need to be taken into account. The lens size is the diagonal diameter of the lens to the tip of the haptics. A recommended PIOL lens size is provided by the lens manufacturer. The PIOL power is related to the curvature of the inner surface of the PIOL. For example, a high powered myopic lens will have an increased radius of curvature of the back surface of the lens resulting in a higher central vault compared to a lower powered lens. PIOL. The PIOL power is also related to the thickness profile of the PIOL, and therefore, PIOLs with different powers react differently when forces are applied. Therefore PIOLs having different powers (assuming all other conditions are identical) will result in different vault outcomes.

The parameters CBID, SPD, STS and STSL will be referred to as the anatomical parameters in the following discussion. The parameters PIOL power and PIOL size will be referred to as the target PIOL parameters.

In one aspect of the invention, a model that depends on the anatomical parameters, the target PIOL parameters and a set of unknown parameters is trained by fitting a data set derived by measuring the number of different patients before and after PIOL implants. In the training mode, the a prior art system for predicting the vault is utilized to minimize the risk of an improperly sized PIOL being implanted into a patient. Once a data set with sufficient statistical accuracy to provide improved accuracy is accumulated, the improvement available with the present system can be realized.

One exemplary model relates the vault of the PIOL to a linear function of the above parameters, e.g.

$$Predicted Vault = a * CBID + b * STSL + c * ( PIOL Power ) + d * ( PIOL size ) + e * SPD$$

The parameters a-e are determined by fitting the observed vault values in the patient group,

While the above-described embodiments utilize a linear model for the relationship between the vault, the measured parameters and desired lens parameters, other functional relationships could be utilized. In addition, the measured parameters could be replaced by other measured quantities that are tightly correlated with the above-described measured parameters.

In some embodiments, the user wishes to find the closest lens size of the desired power from among a finite set of lens sizes. PIOLs are typically manufactured in discrete lens sizes, which differ from one another in steps of approximately 0.5 mm. In one exemplary embodiment, the system of the present disclosure provides a vault value for each PIOL size in some predetermined set of PIOLs.

In other embodiments, the users may wish to know the ideal PIOL size to obtain the desired vault. If sufficient intermediate sizes are found to be of benefit, the results of the current model could be used to alter the choices provided by the manufacturer of the PIOLs.

Once the model is calibrated, the system of the present invention can be practiced on a general purpose computer that is adapted to receive high frequency ultrasound images or on the controller that is included in most high frequency ultrasound devices, In addition, a general purpose computer running the algorithm of the present system can be configured to receive the anatomical measurements and desired PIOL power from a user without the need to extract the anatomical measurements from the ultrasound images. In this case, the anatomical measurements can be provided by the imaging system or by an operator of that system.

Many small eye clinics do not have high frequency ultrasound devices, since many of the measurements normally performed in those clinics utilize optical scanning. Accordingly, a clinic that wishes to utilize the system of the present disclosure may send the patient to a central testing facility that will perform the ultrasound imaging of the patient's eyes and return the scans to the clinic for processing on the clinic's computers. In some embodiments, the imaging center may include the model of the present invention as part of the returned images in the form of a computer readable medium. Alternatively, the controller on the ultrasound imaging device can provide the anatomical measurements required by the model and return that data to the clinic for processing together with the other measurements of the patient's eyes.

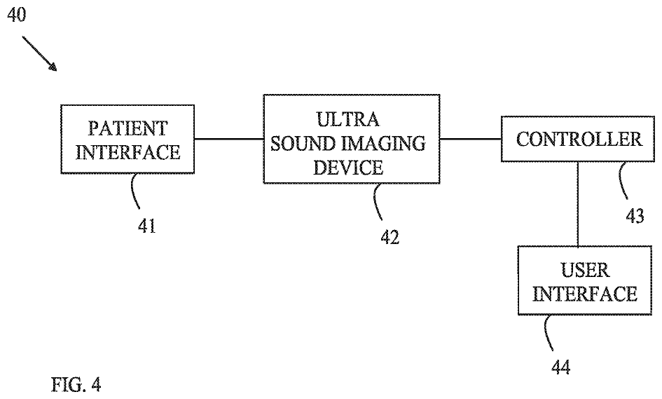

Refer now to FIG. 4, which illustrates one embodiment of an imaging system according to the present disclosure. In system 40, the imaging device forms the images of the patient's eyes using ultrasound imaging device 42 that is coupled to the patient's eye by patient interface 41. The recorded image is processed by controller 43, which extracts the anatomical measurements that depend on the ultrasound image. A user interface 44 allows a user to input additional information such as the PIOL power and possible sizes to controller 43, Controller 43 computes the predicted vault for each lens size requested by the user.

As noted above, the data processing functions of controller 43 can be implemented on a separate data processing system which is adapted to receive ultrasound images from a separate ultrasound imaging device. A user interface of that separate data processing system can be used for inputting the lens properties and outputting the predicted vaults for each desired lens size.

The present invention also includes a computer readable medium that stores instructions that cause a data processing system to execute the method of the present invention. A computer readable medium is defined to be any medium that constitutes patentable subject matter under 35 U.S.C. 101 and excludes any medium that does not constitute patentable subject matter under 35 U.S.C. 101. Examples of such media include non-transitory media such as computer memory devices that store information in a format that is readable by a computer or data processing system.

The above-described embodiments of the present invention have been provided to illustrate various aspects of the invention. However, it is to be understood that different aspects of the present invention that are shown in different specific embodiments can be combined to provide other embodiments of the present invention. In addition, various modifications to the present invention will become apparent from the foregoing description and accompanying drawings. Accordingly, the present invention is to be limited solely by the scope of the following claims.

What is claimed is:

1. A method for operating a data processing system to provide an estimate of a vault value for an implantable collamer lens (ICL) to be inserted in a patient's eye, comprising:
capturing high-frequency ultrasound images of the patient's eye using an ultrasound imaging device, including measuring a diameter of a plane passing through the ciliary body (CBID);
extracting anatomical measurements from the high-frequency ultrasound images including the diameter of the plane passing through the ciliary body (CBID);
receiving parameters specifying a power and a size for the ICL based on the anatomical measurements;
utilizing a calibrated model of a human eye which has been trained on a data set derived from measuring a plurality of different patients before and after ICL implant, to compute an estimated vault value based on the anatomical measurements and the parameters specifying the power and the size for the ICL;
displaying said estimated vault value to a user of said data processing system; and
implanting said ICL in said patient's eye when said estimated vault value is within a predetermined desired range; and
receiving a parameter specifying a different size for said IC when said estimated vault value is not within said desired range.

2. The method for operating the data processing system to provide the estimate of the vault value for the implantable collamer lens (ICL) to be inserted in the patient's eye of claim 1, said causing the data processing system to receive anatomical measurements of the patient's eye further comprising:
the anatomical measurements further comprising sulcus-to-sulcus-lens-rise (STSL) and scotopic pupil diameter (SPD).

3. The method for operating the data processing system to provide the estimate of the vault value for the implantable collamer lens (ICL) to be inserted in the patient's eye of claim 1, said causing the data processing system to receive anatomical measurements of the patient's eye further comprising:
wherein at least one of the anatomical measurements is ultrasound-based.

4. The method for operating the data processing system to provide the estimate of the vault value for the implantable collamer lens (ICL) to be inserted in the patient's eye of claim 1, said utilizing the calibrated model of the human eye to compute the estimated vault value further comprising:
said calibrated model having been calibrated by training the model by fitting a data set derived by measuring a plurality of different patients before and after ICL implant, wherein the data set includes measurement of at least CBID.

5. The method for operating the data processing system to provide the estimate of the vault value for the implantable collamer lens (ICL) to be inserted in the patient's eye of claim 4, said utilizing the calibrated model of the human eye to compute the estimated vault value further comprising:
wherein the data set further includes measurements of sulcus-to-sulcus-lens-rise (STSL) and scotopic pupil diameter (SPD).

6. The method for operating the data processing system to provide the estimate of the vault value for the implantable collamer lens (ICL) to be inserted in the patient's eye of claim 5, said utilizing the calibrated model of the human eye to compute the estimated vault value further comprising:
said calibrated model having been calibrated by training the model to relate a vault value to a linear function of ICL power, ICL size, and at least one of CBID, STSL, and SPD.

7. The method for operating the data processing system to provide the estimate of the vault value for the implantable collamer lens (ICL) to be inserted in the patient's eye of claim 5, said utilizing the calibrated model of the human eye to compute the estimated vault value further comprising:
said calibrated model having been calibrated by training the model to relate a vault value to a function of only CBID, STSL, ICL power, ICL size, and SPD.

8. The method for operating the data processing system to provide the estimate of the vault value for the implantable collamer lens (ICL) to be inserted in the patient's eye of claim 6, said utilizing the calibrated model of the human eye to compute the estimated vault value further comprising:
said calibrated model having been calibrated by training the model to relate the vault value to the function of only CBID, STSL, ICL power, ICL size, and SPD, wherein the function consists of:
$$vault vaule = a * CBID + b * STSL + c * ( ICL Power ) + d * ( ICL size ) + e * SPD ,$$
wherein
the training includes determining parameters a, b, c, d, and e.

9. A system for providing an estimate of a vault value for an implantable collamer lens (ICL) to be inserted in a patient's eye, comprising:
a high-frequency ultrasound imaging device; and
a controller including data processing functions and a calibrated model of a human eye, the controller adapted to receive a high frequency ultrasound image from the high-frequency ultrasound imaging device and for communication with a user interface, wherein the controller is configured to:
receive a high frequency ultrasound image from the high-frequency ultrasound imaging device,
extract a plurality of anatomical measurements of said patient's eye from said image wherein said plurality of anatomical measurements includes CBID,
receive parameters from the user interface specifying at least a power and a size for the ICL, and
utilize the calibrated model of the human eye to compute an estimated vault value based at least on CBID and the parameters specifying the power and the size for the ICL; and
a user interface in communication with the controller configured to receive the estimated vault value and produce an output indicative of the estimated vault value.

10. The system for providing the estimate of the vault value for the implantable collamer lens (ICL) to be inserted in the patient's eye of claim 9, said controller further configured to:
after outputting the estimated vault value to the user interface, receive from the user interface a different size for the ICL; and
utilize the calibrated model of the human eye to compute an updated estimated vault value based at least on CBID, the power of the ICL, and the different size of the ICL; and
output the updated estimated vault value to the user interface.

11. The system for providing the estimate of the vault value for the implantable collamer lens (ICL) to be inserted in the patient's eye of claim 9, said controller further configured to:
extract said plurality of anatomical measurements of said patient's eye from said image, wherein said plurality of anatomical measurements further includes sulcus-to-sulcus-lens-rise (STSL);
utilize said calibrated model of said human eye to compute said estimated vault value, wherein said utilizing is based further on STSL.

12. The system for providing the estimate of the vault value for the implantable collamer lens (ICL) to be inserted in the patient's eye of claim 11, said controller further configured to:
receive at least one additional anatomical measurement from the user interface, wherein said computing of said estimated vault value is based at least on STSL, CBID, the at least one additional anatomical measurement, and the parameters specifying the power and the size for the ICL.

13. The system for providing the estimate of the vault value for the implantable collamer lens (ICL) to be inserted in the patient's eye of claim 12, further comprising:
wherein said at least one additional anatomical measurement comprises scotopic pupil diameter (SPD).

14. The system for providing the estimate of the vault value for the implantable collamer lens (ICL) to be inserted in the patient's eye of claim 13, said controller further configured to:
utilize said calibrated model of said human eye to compute said estimated vault value, wherein said computing of said estimated vault value is based only on CBID, STSL, ICL power, ICL size, and SPD.

15. The system for providing the estimate of the vault value for the implantable collamer lens (ICL) to be inserted in the patient's eye of claim 9, further comprising:
said calibrated model of said human eye having been calibrated by training the model by fitting a data set derived by measuring a plurality of different patients before and after ICL implant, wherein the data set includes measurements of at least CBID.

16. The system for providing the estimate of the vault value for the implantable collamer lens (ICL) to be inserted in the patient's eye of claim 15, further comprising:
wherein the data set further includes measurements of sulcus-to-sulcus-lens-rise (STSL) and scotopic pupil diameter (SPD).

17. The system for providing the estimate of the vault value for the implantable collamer lens (ICL) to be inserted in the patient's eye of claim 16, further comprising:
said calibrated model having been calibrated by training the model to relate a vault value to a linear function of ICL power, ICL size, and at least one of CBID, STSL, and SPD.

18. The system for providing the estimate of the vault value for the implantable collamer lens (ICL) to be inserted in the patient's eye of claim 16, further comprising:
said calibrated model having been calibrated by training the model to relate the vault value to a function of only CBID, STSL, ICL power, ICL size, and SPD.

19. The system for providing the estimate of the vault value for the implantable collamer lens (ICL) to be inserted in the patient's eye of claim 18, further comprising:
said calibrated model having been calibrated by training the model to relate the vault value to the function of only CBID, STSL, ICL power, ICL size, and SPD, wherein the function consists of:
$$vault vaule = a * CBID + b * STSL + c * ( ICL Power ) + d * ( ICL size ) + e * SPD ,$$
the training includes determining parameters a, b, c, d, and e.

20. The system for providing the estimate of the vault value for the implantable collamer lens (ICL) to be inserted in the patient's eye of claim 9, comprising:
the user interface in communication with the controller, wherein the user interface is configured to:
receive input from a user for said parameters specifying at least the power and the size for the ICL;
receive the estimated vault value from the controller; and
display the estimated vault value to the user to determine if said vault value is satisfactory for said patient's eye.
